SEQUENCE LISTING (20 sequences)

SEQ ID NO: 1 - RNA, 43 nt, synthetic construct
  source 1..43: mol_type = other RNA
taatttctactaagtgtagatcttatgcatcagcattgtggga

SEQ ID NO: 2 - RNA, 41 nt, synthetic construct
  source 1..41: mol_type = other RNA
taatttctactaagtgtagatcccccagcgcttcagcgttc

SEQ ID NO: 3 - skipped

SEQ ID NO: 4 - DNA, 24 bp, synthetic construct
  source 1..24: mol_type = other DNA
tggataatgatgcactcaacaaca

SEQ ID NO: 5 - DNA, 26 bp, synthetic construct
  source 1..26: mol_type = other DNA
tgtccatactaatttcactaagttga

SEQ ID NO: 6 - DNA, 40 bp, synthetic construct
  source 1..40: mol_type = other DNA
accattagtttggctgctgtaagagatggttgtgttccct

SEQ ID NO: 7 - DNA, 47 bp, synthetic construct
  source 1..47: mol_type = other DNA
acgtgtgatggtacaacatttactctgcatctacaacctgttggatt

SEQ ID NO: 8 - DNA, 18 bp, synthetic construct
  source 1..18: mol_type = other DNA
tgcatcagcattgtggga

SEQ ID NO: 9 - DNA, 18 bp, synthetic construct
  source 1..18: mol_type = other DNA
aacacaagctttcggcag

SEQ ID NO: 10 - DNA, 22 bp, synthetic construct
  source 1..22: mol_type = other DNA
gaaatttggatctttgtcatcc

SEQ ID NO: 11 - DNA, 41 bp, synthetic construct
  source 1..41: mol_type = other DNA
tgcggccaatgtttgtaatcagccaaggaaattttggggac

SEQ ID NO: 12 - DNA, 39 bp, synthetic construct
  source 1..39: mol_type = other DNA
cgcattggcatggaagtcactttgatggcacctgtgtag

SEQ ID NO: 13 - DNA, 19 bp, synthetic construct
  source 1..19: mol_type = other DNA
ttccttgtctgattagttc

SEQ ID NO: 14 - DNA, 18 bp, synthetic construct
  source 1..18: mol_type = other DNA
accttcgggaacgtggtt

SEQ ID NO: 15 - DNA, 16 bp, synthetic construct
  source 1..16: mol_type = other DNA
tgatgagctggagcca

SEQ ID NO: 16 - DNA, 18 bp, synthetic construct
  source 1..18: mol_type = other DNA
caccctcaatgcagagtc

SEQ ID NO: 17 - DNA, 41 bp, synthetic construct
  source 1..41: mol_type = other DNA
gtgtgaccctgaagactcggttttagccactgactcggatc

SEQ ID NO: 18 - DNA, 45 bp, synthetic construct
  source 1..45: mol_type = other DNA
cctccgtgatatggctcttcgtttttttcttacatggctctggtc

SEQ ID NO: 19 - DNA, 20 bp, synthetic construct
  source 1..20: mol_type = other DNA
atgtggatggctgagttgtt

SEQ ID NO: 20 - DNA, 20 bp, synthetic construct
  source 1..20: mol_type = other DNA
catgctgagtactggacctc

(19) United States
(12) Patent Application Publication
(10) Pub. No.: US 2024/0124947 A1
(43) Pub. Date: Apr. 18, 2024

(54) COMPOSITIONS FOR CORONAVIRUS DETECTION AND METHODS OF MAKING AND USING THEROF

(71) Applicants: Versitech Limited, Hong Kong (CN); Centre for Virology, Vaccinology and Therapeutics Limited, Hong Kong (CN); THE HOSPITAL AUTHORITY, Hong Kong (CN)

(72) Inventors: Kelvin Kai-Wang To, Hong Kong (CN); Siddharth Sridhar, Hong Kong (CN); Kwok-Yung Yuen, Hong Kong (CN); Chik Yan Yip, Hong Kong (CN)

(21) Appl. No.: 18/473,956

(22) Filed: Sep. 25, 2023

Related U.S. Application Data
(60) Provisional application No. 63/376,831, filed on Sep. 23, 2022.

(51) Int. Cl. C12Q 1/70

(52) U.S. Cl. CPC C12Q 1/701; C12Q 2600/16

(57) ABSTRACT

Sequences for detection of SARS-CoV-2 are provided and include example, SEQ ID NOs:4-8. The sequences are useful for amplifying the SARS-CoV-2 nsp8 gene in a sample, using Reverse Transcriptional Loop-Mediated Isothermal Amplification (RT-LAMP) and it is preferably used in combination with a Cas enzyme/sgRNA pair and a reporter nucleic acid.

The disclosed sequences can be use in methods of detecting SARS-CoV-2 nucleic acids in a sample. Generally, the specific gene sequence of SARS-CoV-2 RNA, herein nsp8, is amplified using RT-LAMP. The RT-LAMP products are scanned by the Cas12a-gRNA ribonucleoprotein (RNP) complex. The RNP binds to the specific complementary to gRNA, activating the transcleavage activity of Cas12a. The active Cas12a system cleaves a short ssDNA reporter that is labeled preferably, with a fluorophore and a quencher on either end. Cleavage of the reporter separates the quencher from the fluorophore, and fluorescence that is detectable with the naked eye is generated.

### CROSS-REFERENCE TO RELATED APPLICATIONS

This application claims the benefit of and priority to U.S. Ser. No. 63/376,831, filed Sep. 23, 2022, which is specifically incorporated by reference herein in its entirety.

REFERENCE TO THE SEQUENCE LISTING The Sequence Listing submitted as a text file named “UHK_01210_ST26.xml” created on Aug. 30, 2023, and having a size of 18,178 bytes is hereby incorporated by reference pursuant to 37 C.F.R. § 1.834(c)(1).

### FIELD OF THE INVENTION

This invention is generally in the field of SARS-CoV-2 detection.

### BACKGROUND OF THE INVENTION

The Coronavirus Disease 2019 (COVID-19) pandemic, caused by the Severe Acute Respiratory Syndrome Coronavirus 2 (SARS-CoV-2), remains a public health concern (1-3). With more than 490 million confirmed cases worldwide, it is already one of the largest pandemics in recorded history (4, 5). Prompt COVID-19 diagnosis is a central pillar of pandemic control because this not only guides infection control in community and healthcare settings, but also informs early treatment decisions for at-risk individuals (6-9). Globally, COVID-19 diagnosis mostly relies on antigen tests (usually in a lateral flow format) or nucleic acid amplification tests (NAATs) such as reverse transcription-polymerase chain reaction (RT-PCR) (10). Accurate tests for the diagnosis of SARS-CoV-2, the virus causing coronavirus disease 2019 (COVID-19), are important for timely treatment and infection control decisions. Antigen tests are relatively inexpensive and can be used as point-of-care tests, but suffer from a lack of sensitivity for early COVID-19 diagnosis (11, 12). In contrast, RT-PCR is highly sensitive, but requires centralized testing in a laboratory equipped with trained staff and specialized equipment such as thermocyclers (13-15). Therefore, RT-PCR cannot be conveniently deployed at scale in resource limited settings.

There is still a need improved methods and reagents for detecting SARS-CoV-2, which avoid the need for complex instrumentation and highly trained staff, remain highly sensitive and specific, and reduce costs associated with testing. Such methods will greatly increase the probability of testing by making testing less complex and more affordable.

It is an object of the present invention to provide compositions, methods, and kits for detecting and diagnosing SARS-CoV-2.

It is a further object of the present invention to provide compositions, methods, and kits for detecting and diagnosing SARS-CoV-2 which show improved sensitivity and specificity and reduced costs associated with testing, relative to existing detection methods.

### SUMMARY OF THE INVENTION

Sequences for detection of SARS-CoV-2 are provided and can, for example, include or consist of a sequence of any of SEQ ID NOs:4-8, a sequence having at least 70%, 75%, 80%, 85%, 90%, 95%, 96%, 97%, 98%, or 99% sequence identity thereto, a nucleic acid sequence having 1, 2, 3, 4, 5, 6, 7, 8, 9, or 10 nucleic acid substitution(s), addition(s), deletion(s), or a combination thereof relative thereto, or the reverse complement of any of the foregoing. The sequences are useful for amplifying the SARS-CoV-2 nsp8 gene in a sample, using Reverse Transcriptional Loop-Mediated Isothermal Amplification (RT-LAMP) and it is preferably used in combination with a Cas enzyme/sgRNA pair and a reporter nucleic acid. The reporter nucleic acid is preferably a ssDNA or dsDNA labelled at the 5′ and 3′ ends with a fluorophore/quencher pair, the fluorophore being present at one end (5′/3′) and the quencher at the other end (5′/3′).

The disclosed sequences can be use in methods of detecting SARS-CoV-2 nucleic acids in a sample such as mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood, thus, methods of detecting SARS-CoV-2 in such samples are also provided. The sample can be one that is isolated from a subject that may have been exposed to or is suspected of having SARS-CoV-2. In some embodiments, the sample is processed to expose or isolate nucleic acids from sample before it is subjected to the detection method.

Also disclosed are methods for detecting the presence of SARS-CoV-2 in a sample. The methods use 5 primers designed to amplify conserved the SARS-CoV-2 nsp8 gene in a sample. The disclosed methods include identifying subjects infected with SARS-CoV-2 and/or diagnosing a subject as having COVID 19. The method is an RT-LAMP assay, targeting the SARS-CoV-2 nsp8 gene and it preferably takes advantage of a Cas enzyme/sgRNA pair and a reporter nucleic acid. In a preferred embodiment, the method is a one-step colorimetric RT-LAMP. Generally, the specific gene sequence of SARS-CoV-2 RNA, herein nsp8, is amplified using RT-LAMP. The RT-LAMP products are scanned by the Cas12a-gRNA ribonucleoprotein (RNP) complex. The RNP binds to the specific complementary to gRNA, activating the transcleavage activity of Cas12a. The active Cas12a system cleaves a short ssDNA reporter (for example 8 nt) that is labeled preferably, with a fluorophore and a quencher on either end. The cleavage of the reporter separates the quencher from the fluorophore, resulting in the generation of fluorescence that is detectable with the naked eye. The methods includes the steps of:
(a) contacting the sample with a composition comprising the composition of claim comprising, or consisting essentially of a nucleic acid sequence SEQ ID NO:4; SEQ ID NO:5, SEQ ID NO:5, SEQ ID NO:7, and SEQ ID NO:8, or a nucleic acid sequence having at least 70%, 75%, 80%, 85%, 90%, 95%, 96%, 97%, 98%, or 99% sequence identity to any of the foregoing under conditions sufficient for amplification of the nsp8 gene of SARS-CoV-2, wherein the set of RT-LAMP primers, and
(b) detecting the SARS-CoV-2 nsp8 gene amplification product, thereby detecting presence of SARS-CoV-2 nsp8 in the sample, the method including an optional step before step (b) of contacting the sample from step (a) with a composition comprising a Cas enzyme/sgRNA pair and a reporter nucleic acid.

Assay Kits for RT-LAMP and CRISPR Cas12a-Mediated Ambient Visualization in a Single Tube are disclosed herein. The kits include the compositions disclosed herein including but not limited to and CRISPR Cas12a/sgRNA, reporter nucleic acid, for example labelled ssDNA, RT-LAMP primers.

An assay kit can be prepared to include at least one or two containers. The containers can include liquid reagents, lyophilized reagents and combinations thereof. In embodiments where the one container includes dried reagents, a second container includes a rehydration buffer solution containing primers for RT-LAMP and a ssDNA reporter.

### BRIEF DESCRIPTION OF THE DRAWINGS

FIG. 1 is a workflow of the RT-LAMP-CRISPR assays used in the present study.

FIGS. 2A and 2B are dot plots of the fluorescence readings generated by the in-house developed nsp8 and N gene RT-LAMP-CRISPR assays. 319 specimens tested by RT-LAMP-CRISPR assays, with the dashed threshold lines for nsp8 and N assays (FIG. 2A), and 146 specimens that tested positive by RT-LAMP-CRISPR assays (FIG. 2B). ****, P<0.0001.

FIGS. 3A and 3B are scatterplots of the Correlation of the fluorescence readings generated by the nsp8 (FIG. 3A) and N (FIG. 3B) gene RT-LAMP-CRISPR assays and the Cp values of the LightMix E-gene RT-PCR.

FIG. 4A and FIG. 4B are schematic representations of the multiple sequence alignment using the presently described primer and gRNA sequences and the nsp8 gene sequences of SARS-CoV-2 variants (wild type, Alpha, Beta, Gamma, Delta, Omicron, Lambda and Mu) from different geographical regions. Wild type sequence (NC_045512.2) and the sequences of other variants were obtained from GenBank and GISAID, respectively.

### DETAILED DESCRIPTION OF THE INVENTION

Isothermal NAAT methods such as loop-mediated isothermal amplification (LAMP) obviate the need for a thermocycler by amplifying target nucleic acid at a constant temperature (16). LAMP product detection can be performed using CRISPR-Cas (clustered regularly interspaced short palindromic repeats—CRISPR associated protein) chemistry (17). CRISPR-Cas systems are critical components of bacterial immunity that selectively cleave nucleic acids of invaders like bacteriophages (18). CRISPR effector proteins like Cas12a selectively cleave DNA when activated by guide RNA (gRNA) (19). This feature can be exploited for specific detection of end-products of LAMP.

An assay format for the diagnosis of COVID-19 is described herein, that is based on principles of loop-mediated isothermal amplification (LAMP) and clustered regularly interspaced short palindromic repeat (CRISPR)-Cas chemistry. A major advantage of this assay format is that it does not require expensive equipment to perform, and results can be read visually. This method proved to be fast, easy to perform, and inexpensive. The test compared well against an RT-PCR assay in terms of the ability to detect SARS-CoV-2 RNA in clinical samples. No false-positive test results were observed. The new assay format is ideal for SARS-CoV-2 diagnosis in resource-limited settings.

Several studies demonstrated SARS-CoV-2 detection by RT-LAMP and CRISPR-Cas12a using lateral flow strips (21-23), which involved opening tubes for combining RT-LAMP products with the CRISPR reagent followed by the strip readout. This would increase the chance of amplicon contamination. To reduce the risk of contamination, the disclosed assay is in a one-tube format. Another advantage of the disclosed methods is that it reduces the cost of each reaction without sacrificing the sensitivity of the assay, and therefore we attempted to reduce the amount of RT-LAMP reagents compared to those used in other studies (22, 24, 25). The assay disclosed herein is rapid, sensitive, specific, affordable, and user-friendly. In addition, it does not require thermocyclers, enabling it to be deployed at scale even in resource-limited settings.

### I. Definitions

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclose

The term “amplification” as used herein refers to increasing the number of copies of a nucleic acid molecule, such as a gene or fragment of a gene, for example at least a portion of the SARS-CoV-2 RNA. The products of an amplification reaction are called amplification products. An example of in vitro amplification is loop-mediated isothermal amplification.

The terms “complement”, “complementary” or “complementarity” as used herein with reference to polynucleotides (i.e., a sequence of nucleotides such as an oligonucleotide or a target nucleic acid) refer to the Watson/Crick base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in naturally occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence and can also be a cDNA.

The term “conditions sufficient for” as used herein in connection with the disclosed methods, refers to any environment that permits the desired activity, for example, that permits specific binding or hybridization between two nucleic acid molecules or that permits reverse transcription and/or amplification of a nucleic acid. Such an environment may include, but is not limited to, particular incubation conditions (such as time and/or temperature) or presence and/or concentration of particular factors, for example in a solution (such as buffer(s), salt(s), metal ion(s), detergent(s), nucleotide(s), enzyme(s), etc.).

The term “contact” as used herein in connection with the disclosed methods refers to placement in direct physical association; for example, in solid and/or liquid form. For example, contacting can occur in vitro with one or more primers and/or probes and a biological sample (such as a sample including nucleic acids) in solution.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of double-stranded DNA (dsDNA). A “reverse primer” anneals to the sense-strand of dsDNA.

Primers are typically at least 10, 15, 18, or 30 nucleotides in length or up to about 100, 110, 125, or 200 nucleotides in length. In some embodiments, primers are preferably between about 15 to about 60 nucleotides in length, and most preferably between about 25 to about 40 nucleotides in length. In some embodiments, primers are 15 to 35 nucleotides in length. There is no standard length for optimal hybridization or polymerase chain reaction amplification. An optimal length for a particular primer application may be readily determined in the manner described in H. Erlich, PCR Technology, PRINCIPLES AND APPLICATION FOR DNA AMPLIFICATION, (1989).

As used herein, the term “sample” refers to in vitro as well as clinical samples obtained from a patient. In preferred embodiments, a sample is obtained from a biological source (i.e., a “biological sample”), such as tissue or bodily fluid collected from a subject. Sample sources include, but are not limited to, mucus, sputum (processed or unprocessed), bronchial alveolar lavage (BAL), bronchial wash (BW), blood, bodily fluids, cerebrospinal fluid (CSF), urine, plasma, serum, or tissue (e.g., biopsy material), nasopharyngeal aspirate, nasopharyngeal swab, throat swab, and other discussed herein and otherwise known in the art.

“Sensitivity” as used herein, is a measure of ability of a detection assay to detect the presence of a target sequence directly or indirectly (e.g., a SARS-CoV-2 viral sequence) in a sample.

“Specificity,” as used herein, is a measure of the ability of a detection assay to distinguish a truly occurring target sequence (e.g., a SARS-CoV-2 viral sequence) from other closely related sequences (e.g., other human-pathogenic coronaviruses and respiratory pathogens). It is the ability to avoid false positive detections.

The terms “target nucleic acid” or “target sequence” or “target segment” as used herein refer to a nucleic acid sequence of interest to be detected and/or quantified in the sample to be analyzed. Target nucleic acid may be composed of segments of a genome, a complete gene with or without intergenic sequence, segments or portions of a gene with or without intergenic sequence, or sequence of nucleic acids to which probes or primers are designed to hybridize. Target nucleic acids may include a wild-type sequence(s), a mutation, deletion, insertion or duplication, tandem repeat elements, a gene of interest, a region of a gene of interest or any upstream or downstream region thereof. Target nucleic acids may represent alternative sequences or alleles of a particular gene. Target nucleic acids may be derived from genomic DNA, cDNA, or RNA.

### II. Compositions

The disclosed compositions include primers designed to recognize distinct target sequences on a template strand. The SARS-CoV-2 genome includes 5′-untranslated region (5′-UTR), open reading frame 1a/b encoding non-structural proteins (nsp) for replication, structural proteins including spike (blue box), envelop (orange box), membrane (red box), and nucleocapsid (cyan box) proteins, accessory proteins (purple boxes) such as orf 3, 6, 7a, 7b, 8 and 9b in the 2019-nCoV (HKU-SZ-005b) genome, and the 3′-untranslated region (3′-UTR). Chan, et al., Emerg Microbes and Infections 9(1): 221-2361(2020).

The designed primers target the nsp8 gene of SARS-CoV-2. Such primers bind only to these sequences which allows for high specificity. Out of the primers involved, two of them are “inner primers” (FIP and BIP), designed to synthesize new DNA strands. The outer primers (F3 and B3) anneal to the template strand and also generate new DNA.

The RT-LAMP reaction is carried out in a mixture including a suitable buffer (such as a phosphate buffer or Tris buffer). Isothermal Amplification Buffers are commercially available from suppliers such as the New England Biolabs Inc. (catalog #B0537S). The buffer may also include additional components, such as salts (such as KCl or NaCl, magnesium salts (e.g., MgCl₂ or MgSO₄), ammonium (e.g., (NH₄)₂SO₄)), detergents (e.g., TRITON-X100), or other additives (such as betaine or dimethylsulfoxide). The buffer or reaction mixture also includes nucleotides or nucleotide analogs. In some forms, an equimolar mixture of dATP, dCTP, dGTP, and dTTP (referred to as dNTPs) is included, for example about 5-15 mM dNTPs (such as about 5-8 mM dNTPs).

Primers

These primers are accompanied by a DNA polymerase, which can aid strand displacement and can release the newly formed DNA strands. Preferred primers for SARS-CoV-2 nsp8 gene detection are shown below in Table 1, in the 5′-3′ direction.

TABLE 1
nsp8 Primers used for RT-LAMP reactions

| Primer | Sequence (5′-3′) |
|---|---|
| SARS-COV-2_ | TGGATAATGATGCACTCAACAACA (SEQ ID |
| nsp8_F3 | NO: 4) |
| SARS-COV-2_ | TGTCCATACTAATTTCACTAAGTTGA (SEQ |
| nsp8_B3 | ID NO: 5) |
| SARS-COV-2_ | ACCATTAGTTTGGCTGCTGTAAGAGATGGT |
| nsp8_FIP | TGTGTTCCCT (SEQ ID NO: 6) |
| SARS-COV-2_ | ACGTGTGATGGTACAACATTTACTCTGC |
| nsp8_BIP | ATCTACAACCTGTTGGATT (SEQ ID NO: 7) |
| SARS-COV-2_ | TGCATCAGCATTGTGGGA (SEQ ID NO: 8) |
| nsp8_LB | |

Thus, useful primers include SEQ ID NO:4; SEQ ID NO:5, SEQ ID NO:5, SEQ ID NO:7, and SEQ ID NO:8, or a nucleic acid sequence having at least 70%, 75%, 80%, 85%, 90%, 95%, 96%, 97%, 98%, or 99% sequence identity to any of the foregoing.

DNA Polymerase

A DNA polymerase with strand displacement activity is also included in the reaction mixture. Exemplary DNA polymerases include Est DNA polymerase, first 2.0 DNA polymerase, Bst 2.0 WarmStart™ DNA polymerase (New England Biolabs, Ipswich, Mass.), Phi29 DNA polymerase, Bsu DNA polymerase, Omni Amp™ DNA polymerase (Lucigen, Middleton, Mich.), Taq DNA polymerase, Vent_{R}® and Deep Vent_{R}® DNA polymerases (New England Biolabs), 9° Nₘ™ DNA polymerase (New England Biolabs), Klenow fragment of DNA polymerase I, PhiPRD1 DNA polymerase, phage M2 DNA polymerase, T4 DNA polymerase, and T5 DNA polymerase.

Commercially available DNA polymerase mixtures as available, such as the WarmStart Colorimetric LAMP 2× Master Mix is an optimized formulation of Bst 2.0 WarmStart DNA Polymerase and WarmStart RTx in a special low-buffer reaction solution containing a visible pH indicator for rapid and easy detection of Loop-Mediated Isothermal Amplification (LAMP) and RT-LAMP reactions. This system is designed to provide a fast, clear visual detection of amplification based on the production of protons and subsequent drop in pH that occurs from the extensive DNA polymerase activity in a LAMP reaction, producing a change in solution color from pink to yellow P 2× Master Mix (New England Biolabs, Ipswich, USA) can be used.

Thus, in some forms, one or more of the disclosed nucleic acids for detecting SARS-CoV-2 are in a composition containing additional reagents for amplification reactions, such as buffer, enzymes (such as reverse transcriptase and DNA polymerase), dNTPs, or other reagents. In some forms, one or more of the disclosed nucleic acids for detecting SARS-CoV-2 are in a composition including all of the components required for the reaction except the sample (and water/buffer, if the reagents are supplied in dried or lyophilized form).

Cas Enzyme/sgRNA

CRISPR-Cas was first identified as part of the immunological system of bacteria¹¹⁵⁻¹¹⁷. This system is naturally composed of one or several CRISPR-associated (Cas) proteins and a genomic region termed Clustered Regularly Interspaced Short Palindromic Repeats (CRISPR) which memorizes previous pathogen attacks. For engineering purposes, the CRISPR-Cas system has been adapted such that only a single guide RNA molecule and, generally, only one Cas protein are needed to bind and, in some cases, cleave a nucleic acid target. Notably, the system only binds and activates its catalytic function if the target contains a sequence that is complementary to that defined in the spacer region of the guide RNA molecule.

RNA-detection methods based on CRISPR-Cas systems usually work in solution and their mechanism of action can be summarized in four steps a) sample processing (i.e. RNA extraction) and target amplification, b) Ribonucleoprotein (RNP) complex formation comprising the CRISPR associated protein (Cas) and a guide RNA, c) Target recognition followed by a conformational change of the RNP complex and d) Collateral cleavage of a fluorescence-quenched reporter molecule. Thus, the CRISPR-Cas system relies on two main components: a guide RNA (gRNA) and CRISPR-associated (Cas) nuclease, which together form a ribonucleoprotein (RNP) complex. The gRNA is a specific RNA sequence that recognizes the target DNA region of interest and directs the Cas nuclease there for editing. The gRNA is made up of two parts: crispr RNA (crRNA), a 17-20 nucleotide sequence complementary to the target DNA, and a tracr RNA (transactivating crRNA), which serves as a binding scaffold for the Cas nuclease. crRNAs and tracrRNAs exist as two separate RNA molecules in nature. By contrast, sgRNA (single guide RNA) is single RNA molecule that contains both a custom-designed short crRNA sequence fused to the scaffold tracrRNA sequence. sgRNA can be synthetically generated or made in vitro or in vivo from a DNA template. The CRISPR-associated protein is a non-specific endonuclease. It is directed to the specific DNA locus by a sgRNA, where it makes a double-strand break.

sgRNA design implements finding target sites in the genome by scanning a protospacer adjacent motif (PAM) sequence (like 5′-NGG-3′ for SpCas9). Once the target gene and Cas nuclease have been selected, known methods can be used to design the specific guide RNA sequence. Tools for CRISPR/Cas9 sgRNA design are known in the art and are reviewed in Cui, et al., Interdisciplinary Sciences: Computational Life Sciences, 10:455465 (201.8), Methods for making sgRNA are known and include synthetically generating the sgRNA or by making the guide in vivo or in vitro, starting from a DNA template. One method involves expressing the guide RNA sequence in cells from a transfected plasmid. In this method, the sgRNA sequence is cloned into a plasmid vector, which is then introduced into cells. The cells use their normal RNA polymerase enzyme to transcribe the genetic information in the newly introduced DNA to generate the sgRNA. Another method for making sgRNA, termed in vitro transcription (INT), involves transcribing the sgRNA from the corresponding DNA sequence outside the cell. First, a DNA template is designed that contains the guide sequence and an additional RNA polymerase promoter site upstream of the sgRNA sequence. The sgRNA is then transcribed using kits that contain reagents and recombinant RNA polymerase. Kits are commercially available, for example, TranscriptAid T7 High Yield Transcription Kit (ThermoFisher Scientific).

Specifically, Cas12a's crRNA is programmed to recognize an exact double-stranded (ds) DNA target sequence that is accompanied by a T-rich protospacer adjacent motif (PAM). The single target recognition induces a conformational change in Cas12a RuvC active site, hydrolyzing a 5′-phosphodiester linkage distal from the PAM sequence. The distal cleavage product is then released activating Cas12a for indiscriminate cleavage of all fluorescent (F) and quencher (Q) labeled single-stranded (ss) DNA in the environment, generating a fluorescence readout. ssDNA reporters and double stranded (ds) DNA reporters for Cas 12a of varying lengths are known in the art. Smith, et al., ACS Synth Biol. 2021 10(7): 1785-1791.

Exemplary gRNAs specifically targeting the RT-LAMP amplicons of nsp8 and the ssDNA reporter are shown in Table 4.

Other Cas enzymes that can be used in the disclosed methods include Cas3, Cas9, Cas10, Cas13, Cas14, mCas13 (miniature Cas13), etc., all of which are known in the art and methods of generating sgRNA to target a gene of interest for use with a specific Cas enzyme are also known in the art. Non-limiting examples of suitable Cas enzymes are described in Ali Z, et al. 2020 Virus Res. Vol. 288: Article number 198129; Mahas A, et al. 2021, ACS Synth Biol. Vol. 10(10): pages 2541-2551, Santiago-Frangos A, et al. 2021 Cell Rep Med. Vol. 2(6): article number 100319, and Yoshimi K, et al. 2022, iScience Vol. 25(2): article number 103830.

### III. Methods of Using

The disclosed compositions can be used to detect the presence for SARS-CoV-2 in a sample, by Loop-mediated isothermal amplification (LAMP), preferably, a reverse-transcription step to allow the detection of RNA (RT-LAMP). The disclosed methods include identifying subjects infected with SARS-CoV-2 and/or diagnosing a subject as having COVID 19. A subject is diagnosed as having COVID 19 where the samples is obtained from a subject presenting with one or more symptoms associated with COVID 19, including, but not limited to, fever, dry cough, fatigue, loss of appetite, loss of smell, loss of taste, body aches, shortness of breath, muscle weakness, tingling or numbness in the hands and feet, dizziness, confusion, delirium, seizures, stroke, nausea, vomiting, diarrhea, and abdominal pain or discomfort associated, and the sample is contacted with compositions comprising a nucleic acid sequence SEQ ID NO:4; SEQ ID NO:5, SEQ ID NO:5, SEQ ID NO:7, and SEQ ID NO:8, or a nucleic acid sequence having at least 70%, 75%, 80%, 85%, 90%, 95%, 96%, 97%, 98%, or 99% sequence identity to any of the foregoing.

Exemplary samples include such as mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood. Preferred samples include Nasopharyngeal swabs/aspirates, sputa/deep throat saliva, throat swabs.

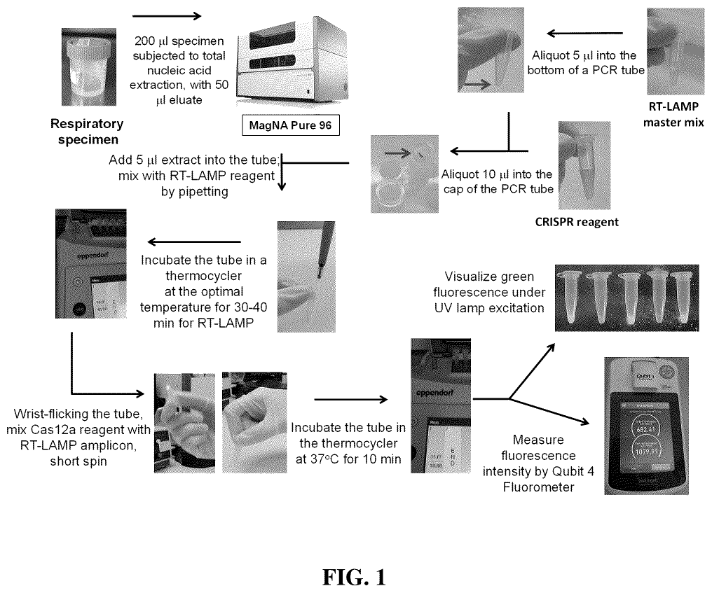

As exemplified herein, evaluation using clinical samples from COVID-19 patients and patients with other respiratory virus infections showed that this COVID-19-LAMP assay is highly sensitive and specific. The disclosed methods can detect the presence of SARS-CoV-2 in a sample with a sensitivity of at least about 90%, such as a sensitivity of 90%, 95%, 98%, etc. and a specificity of at least 90%, 5%, or even 100%. The result can be unambiguously visualized by the naked eye and interpreted by any person, with short turn-around-time (including sample extraction) of 45-105 min depending on the viral load (FIG. 1).

A. Sample Preparation

Samples include respiratory specimens such as nasopharyngeal swabs/aspirates, sputa/deep throat saliva, throat swabs, in a transport medium or in viral inactivation collection tubes. Specimens should be kept at 2-4° C. or extracted within four hours of testing.

Sample preparation methods for detection of virus in biological samples are known in the art and exemplified below. For example, in some embodiments a kit such as QIAamp Viral RNA Mini Kit (QIAGEN, #52906, 250 reactions) is used as the RNA extraction system. For instance, rapid nucleic acid preparation can be performed using a commercially available kit (such as kits and/or instruments from Qiagen (such as QiaAmpO, DNEasy® or RNEasy® kits), Roche Applied Science (such as MagNA Pure kits and instruments), Thermo Scientific (KingFisher mL), bioMerieux (Nuclisens® NASBA Diagnostics), or Epicentre (Masterpure™ kits)). The volume of the specimens used for extraction and the elution volume depend on the specimen type and the available amount of the specimen.

In some forms RNA is not extracted from the sample i.e., the sample is subject to method steps without the initial step of extracting RNA from the sample. In these embodiments, a <5 ml, for example about 4 ml. 3 ml, 2 ml or 1 mL of the NPS specimen/sample without heat pretreatment and a <5 ml, for example about 4 ml. 3 ml, 2 ml or 1 mL of specimen/sample with heat pretreatment (98° C. for 5 min) are preferred conditions for the disclosed RT-LAMP-CRISPR assays. In some forms a lysis buffer/proteinase K are not used.

B. Methods of Detection

The general set up and overall operation of the assay for the detection of SARS-Co-V-2 is disclosed in FIG. 1. Generally, the specific gene sequence of SARS-CoV-2 RNA, herein nsp8, is amplified using RT-LAMP. The RT-LAMP products are scanned by the Cas12a-gRNA ribonucleoprotein (RNP) complex. The RNP binds to the specific complementary to gRNA, activating the transcleavage activity of Cas12a. The active Cas12a system cleaves a short ssDNA reporter (8 nt) that is labeled preferably, with a fluorophore and a quencher on either end. The cleavage of the reporter separates the quencher from the fluorophore, resulting in the generation of fluorescence.

i. RT-LAMP

The disclosed method uses reverse transcription coupled loop-mediated isothermal amplification (RT-LAMP) methods, to detect the presence of a pathogen of interest, preferably viral infections, for example, the presence of SARS-Co-V-2 RNA in a sample.

Loop-mediated isothermal amplification (LAMP) is a single-tube technique for the amplification of DNA, which may be combined with a reverse-transcription step to allow the detection of RNA (RT-LAMP). RT-LAMP requires primers, a reverse transcriptase enzyme, and a DNA polymerase enzyme having strand displacement activity for the amplification of RNA. Similar to RT-PCR, conventional RT-LAMP requires a reverse transcriptase enzyme to synthesize complementary DNA (cDNA) from RNA sequences. This cDNA can then be amplified using DNA polymerase.

RT-LAMP is desirable because of the relatively low reaction temperature and no need for thermocycling equipment necessary for other methods like PCR. In conventional LAMP, four specially designed primers can recognize distinct target sequences on a template strand. Such primers bind only to these sequences which allows for high specificity. Out of the four primers involved, two of them are “inner primers” (FIP and BIP), designed to synthesize new DNA strands. The outer primers (F3 and B3) anneal to the template strand and also generate new DNA. These primers are accompanied by a DNA polymerase which can aid strand displacement and can release the newly formed DNA strands.

The BIP primer (in conventional methods, accompanied by a reverse transcriptase enzyme), can initiate the process by binding to a target sequence on the 3′ end of an RNA template and synthesizing a copy DNA strand. The B3 primer can also bind the 3′ end and along with a polypeptide having DNA polymerase activity (e.g., a mutant polymerase described herein) can simultaneously create a new cDNA strand while displacing the previously made copy. The double stranded DNA containing the template strand is no longer needed.

At this point, the single stranded copy can loop at the 3′ end as it binds to itself. The FIP primer can bind to the 5′ end of this single strand and accompanied by a polypeptide having DNA polymerase activity, can synthesize a complementary strand. The F3 primer, with DNA polymerase, can bind to this end and can generate a new double stranded DNA molecule while displacing the previously made single strand.

This newly displaced single strand can act as the starting point for a LAMP cycling amplification. The DNA can have a dumbbell-like structure as the ends fold in and self-anneal. This structure can become a stem-loop when the FIP or BIP primer once again initiates DNA synthesis at one of the target sequence locations. This cycle can be started from either the forward or backward side of the strand using an appropriate primer. Once this cycle has begun, the strand can undergo self-primed DNA synthesis during the elongation stage of the amplification process. This amplification can take place in about an hour, under isothermal conditions between about 60-65° C., preferably, about 60° C.

ii. Detection of Amplification Products

In the foregoing methods (e.g., one-step RT-PCR, RT-LAMP) following the amplification reaction, i.e., RT-LAMP, the amplification product(s) can be detected by any suitable method.

For example, disclosed is method of detecting presence of a nucleic acid in a sample by combining the sample and plurality of primers specific to the nucleic acid with a disclosed composition under conditions sufficient for amplification of the nucleic acid, and detecting the nucleic acid amplification product, thereby detecting presence of the nucleic acid in the sample.

The detection method may be quantitative, semi-quantitative, or qualitative. The particular nucleic acid (e.g., viral nucleic acid) may be determined in some cases by the band pattern observed on gel electrophoresis.

Amplification products can also be detected using a colorimetric assay, such as with an intercalating dye (for example, propidium iodide, SYBR green, GelRed™, or GelGreen™ dyes).

Amplification products can also be detected with a metal ion sensitive fluorescent molecule (for example, calcein, which is a fluorescence dye that is quenched by manganese ions and has increased fluorescence when bound to magnesium ions).

In some embodiments, a sample is identified as containing a viral nucleic acid (for example, the sample is “positive” for the virus) if one or more amplifications products are detected (e.g., by any suitable quantitative, semi-quantitative, or qualitative approach). In some embodiments, a sample is identified as containing a viral nucleic acid (for example is “positive” for the virus) if an increase in fluorescence is detected compared to a control (such as a no template control sample or a known negative sample).

In a preferred embodiment, the RT-LAMP amplification product is detected using a Cas12a/sgRNA system as disclosed herein and exemplified below. In this embodiment fluorescence is used for the quantitative detection of one or more amplification products. Generally, this relies on use of a detection probe containing a fluorophore moiety and a quencher moiety, positioned in such a way that the hybridized state of the probe can be distinguished from the unhybridized state of the probe by an increase in the fluorescent signal from the nucleotide. The fluorophore and quencher molecules are incorporated into the probe in sufficient proximity such that the quencher quenches the signal of the fluorophore molecule when the probe is hybridized to its recognition sequence, fluorophore moieties and quencher moieties that can be used to label a ssDNA reporter for use in the disclosed methods are known in the art, and exemplified herein using a ssDNA (8 nt) reporter dually labeled with a fluorophore (6-carboxyfluorescein, 6-FAM) at the 5′ end and a quencher (Iowa Black® fluorescence quencher, IABk FQ (IDT integrated DNA Technologies) at the 3′ end. The ssDNA reporter serves as a substrate or the trans-cleavage activity of Cas12a. Other fluorophore/quencher pairs are known in the art. For example, the fluorophore is FAM (fluorescein), TET (tetrachloro flourescein), HEX (hexachlorofluorescein), JOE (5′-Dichloro-dimethoxy-fluorescein), 6-JOE (6-carboxy-4′,5′-dichloro-2′,7′-dimethoxyfluorescein); MAX, Cy3, or TAMRA and the quencher is IBFQ, a BLACK HOLE quencher, for example, attached to the 5′ end or the 3′ end of reporter nucleic acid i.e., the ssDNA or dsDNA. Tetramethylrhodamine (TMR) is an important fluorophore for preparing, protein conjugates. Under the name TAMRA, the carboxylic acid of TMR has also achieved prominence as a dye for oligonucleotide labeling and automated DNA sequencing applications, 5-(and -6)-carboxytetramethylrhodamine, succinimidyl ester is the amine-reactive, mixed isomer form of TAMRA. Single isomer forms of 5-TAMRA SE (Thermofisher Cat no. C-2211) and 6-TAMRA SE (Thermofisher Cat. no. C-6123) are also available. Exemplary quenchers include BHQ (Black Hole Quencher)1, BHQ2, or BHQ3.

In certain embodiments, the fluorophore is ROX, Texas Red, Cy5, or Cy5.5 and the quencher is IBRQ or BHQ2 (WO 2013/033436). In some embodiments, the fluorophore is FAM, TET, or VIC and the quencher is TAMRA. The Chemical structure for VIC phosphoramidite is shown below.

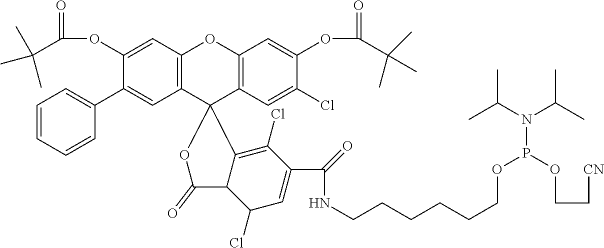 [SMILES: CC(C)N(C(C)C)P(OCCC#N)OCCCCCCNC(=O)C1=CC(Cl)C2C(=O)OC3(C2=C1Cl)c1cc(Cl)c(OC(=O)C(C)(C)C)cc1Oc1cc(OC(=O)C(C)(C)C)c(-c2ccccc2)cc13]

In some embodiments, that fluorophore is FAM, NED, TET, or VIC, and the quencher is MGB. In some embodiments, that fluorophore is ABY, FAM, JUN or VIC, and the quencher is QSY7. In some embodiments, that fluorophore is Yakima Yellow or SUN, and the quencher is Iowa Black FQ. In some embodiments, the fluorophore TEX615 or TYE and the quencher is Black RQ-Sp or BHQ2.

The sample and RT-LAMP primer set(s) are contacted under conditions sufficient for amplification of a viral nucleic acid(s), producing an amplification product. The sample is contacted with the set(s) of RT-LAMP primers at a concentration sufficient to support amplification of the nsp8 gene of the SARS-CoV-2. The RT LAMP reaction is carried out in a mixture including a suitable buffer (such as a phosphate buffer or Tris buffer). The buffer may also include additional components, such as salts (such as KCl or NaCl, magnesium salts (e.g., MgCl₂ or MgSO₄), ammonium (e.g., (NH₄)₂SO₄)), detergents (e.g., TRITON-X100), or other additives (such as betaine or dimethylsulfoxide). The buffer or reaction mixture also includes nucleotides or nucleotide analogs. In some forty's, equimolar mixture of dATP, dCTP, dGTP, and dTTP (referred to as dNTPs) is included, for example about 0.5-5 mM dNTPs (such as about 1-3 mM dNTPs).

The methods use 5 primers, which is targeting the highly conserved nsp8 gene of SARS-CoV-2.

In some embodiments, the optimal RT-LAMP reaction conditions using the nsp8 gene as a target, include primer concentrations:
outer primer F3, B3: at a final concentration between about 0.1 μM to about 0.4 μM.
inner primer FIP, BIP: at a final concentration between about 0.8 μM to about 3.2 μM. and
LoopB (LB): at a final concentration between about 0.2 μM to about 1.6 μM.

A temperature between about 60° C. and 65° C. is preferred and reaction time (30 min, 40 min, 60 min, preferably, about 40 min). As exemplified herein, each 10 μL RT-LAMP reaction can contain 0.4 μL of nuclease-free water, 1 μL of 10× isothermal Amplification Buffer (NEB, USA), 0.6 μL of 100 mM MgSO₄ (NEB), 1.4 μL of 10 mM dNTP solution mix, 1 μL of 10×LAMP Primer Mix, 0.4 μL of Bst 2.0 WarmStart DNA Polymerase (8000 units/mL) (NEB), 0.2 μL of WarmStart RTx Reverse Transcriptase (15,000 units/mL) (NEB) and 5 μL of TNA as the template.

### IV. Kits

Assay Kits for RT-LAMP and CRISPR Cas12a-Mediated Ambient Visualization in a Single Tube are disclosed herein.

An assay kit can be prepared to include at least one or two containers for example, a 0.2 mL PCR tube and a vial. The PCR tube can in one embodiment, contain dried reagents for the assay, and the vial can include rehydration buffer solution containing optionally primers and a reporter. The vial of rehydration buffer preferably contains the primers for RT-LAMP (for example, SEQ ID NOs: 4-8), and the ssDNA reporter for the Cas12a-mediated detection.

In one embodiment, the mixture of the RT-LAMP reaction reagents, except the primers, is added to the bottom of the PCR tube. This mixture can include dNTP, isothermal amplification buffer, MgSO₄, RNase inhibitor, reverse transcriptase, and DNA polymerase, and a suitable stabilizing agent such as D-(+)-trehalose dihydrate, mannitol, sucrose, etc. Agents for stabilizing biomolecules during lyophilization are known in the art.

The RNP complex for the Cas12a-mediated reaction, MgSO₄, and Tris-HCl are added inside the cap of the tube. The tube is placed in a vacuum desiccator for 2 h to dry the reagents. After the reagents are dried, the tube is capped and stored at 4° C. until use.

### Examples

Methods

Viruses, Clinical Specimens, and Proficiency Testing Samples for Evaluation

For analytical sensitivity evaluation, two-fold serial dilutions of total nucleic acid (TNA) extracted from SARS-CoV-2 Q Control 01 with the target concentration of 10,000 dC/mL (Qnostics, UK) were used. Triplicates were performed for each dilution in two independent experiments.

For analytical specificity evaluation, TNA extracted from the culture isolates of human coronaviruses (SARS-CoV-1, MERS-CoV, HCoV-OC43, HCoV-229E and HCoV-NL63), influenza A viruses (A[H1N1]pdm09 and H3N2), influenza B virus, influenza C virus, human adenovirus, rhinovirus, respiratory syncytial virus, human metapneumovirus, human parainfluenza virus types 1-4 were used. For HCoV-HKU1, TNA was extracted from a patient's specimen^{29, 30, 42-44}.

For diagnostic performance evaluation, archived respiratory specimens that were previously tested by real-time RT-PCR assay of the LightMix® SarbecoV E-gene kit (TIB MolBiol, Germany) from year 2020-2021 were included in this study. A total of 319 clinical specimens (149 nasopharyngeal and 170 posterior oropharyngeal saliva) from 319 hospitalized patients (male:female=149:170; median age: 35 years; range: 9 months-103 years) with suspected COVID-19 were selected for SARS-CoV-2 RNA detection. The SARS-CoV-2 positive specimens were subjected to lineage identification by nanopore sequencing⁴⁵. In addition to clinical specimens, 11 proficiency testing samples from the CAP and QCMD with different concentrations of SARS-CoV-2 RNA or negative for SARS-CoV-2 RNA were also evaluated.

This study was approved by the Institutional Review Board of the University of Hong Kong/Hospital Authority Hong Kong West Cluster (HKU/HA HKW IRB) (UW 20-224). Since archived specimens were used, written informed consent was waived.

Reverse Transcription Loop-Mediated Isothermal Amplification (RT-LAMP)

TNA extraction was performed using MagNA Pure 96 extraction system (Roche, Switzerland) according to manufacturer's instruction as we described in previous studies⁸. Briefly, 200 μL of each sample was mixed with MagNA Pure 96 External Lysis Buffer (Roche). After extraction, TNA was recovered in 50 μL of elution buffer and then kept at −80° C. until use.

Before preparing a master mix for RT-LAMP assay, 10×LAMP Primer Mix was prepared (Tables 1 and 2).

TABLE 2
10X LAMP Primer Mix for nsp8

| Reagent | Volume (μL) |
|---|---|
| Nuclease-free water | 60 |
| SARS-CoV-2_nsp8_FIP (100 μM) | 16 |
| SARS-CoV-2_nsp8_BIP (100 μM) | 16 |
| SARS-CoV-2_nsp8_F3 (100 μM) | 2 |
| SARS-CoV-2_nsp8_B3 (100 μM) | 2 |
| SARS-CoV-2_nsp8_LB (100 μM) | 4 |

TABLE 3
10X LAMP Primer Mix for N assay

| Reagent | Volume (μL) |
|---|---|
| Nuclease-free water | 48 |
| SARS-CoV-2_N_FIP (100 μM) | 16 |
| SARS-CoV-2_N_BIP (100 μM) | 16 |
| SARS-CoV-2_N_F3 (100 μM) | 2 |
| SARS-CoV-2_N_B3 (100 μM) | 2 |
| SARS-CoV-2_N_LF (100 μM) | 8 |
| SARS-CoV-2_N_LB (100 μM) | 8 |

A RT-LAMP reagent mixture (10 μL) contained 0.4 μL of nuclease-free water, 1 μL of 10× isothermal Amplification Buffer (NEB, USA), 0.6 μL of 100 mM MgSO4 (NEB), 1.4 μL of 10 mM dNTP solution mix, 1 μL of 10×LAMP Primer Mix, 0.4 μL of Bst 2.0 WarmStart DNA Polymerase (8000 units/mL) (NEB), 0.2 μL of WarmStart RTx Reverse Transcriptase (15,000 units/mL) (NEB) and 5 μL of TNA as the template. RT-LAMP reactions were performed at 60° C. for 40 minutes for nsp8 assay and 62° C. for 30 minutes for N assay.

A comparison of improved reaction conditions with the method RT-PCT method disclosed in Pang, et al., which uses the N gene and E genes as targets, is shown in Table 4 below.

TABLE 4
Comparison of Reaction Conditions between
Present Method and Pang et al.

| | | | Present | |
|---|---|---|---|---|
| | Pang et al.²² | | Method | |
| Vol of RT-LAMP reaction solution- | 25 | μL | 10 | μL |
| dNTP | 1.4 | mM | 1.4 | mM |
| F3 and B3 | 0.2 | μM each | 0.2 | μM each |
| FIP and BIP | 1.6 | μM each | 1.6 | μM each |
| LB | 0.8 | μM | 0.4 | μM |
| LF | 0.8 | μM | N/A | |
| WarmStart RTx reverse transcriptase | 7.5 | units | 3 | units |
| Bst 2.0 DNA polymerase | 8 | units | 3.2 | units |
| MgSO₄ | 6 | mM | 6 | mM |
| 10X Isothermal Amplification Buffer | 2.5 | μL | 1 | μL |
| Sample | 1-10 | μL | 5 | μL |

CRISPR Cas12a-Based Fluorescence Detection

Cas12a trans-cleavage assay was performed as previously described by Pang et al²², albeit with superior modifications to the prior RT-LAMP assay, which advantageously reducing the amount of RT-LAMP reagents while maintaining the sensitivity of the assay. Briefly, the reagent mixture (10 μL) contained 1.7 μL of nuclease-free water, 2 μL of 10×NEBuffer r2.1, 1 μL of 10 μM of guide RNA (gRNA), 0.8 μL of 10 μM of EnGen Lba Cas12a (Cpf1) (NEB), 0.5 μL of 100 μM ssDNA reporter and 4 μL of 100 mM MgSO₄. The gRNAs specifically targeting the RT-LAMP amplicons of nsp8 and N genes and the ssDNA reporter were designed and synthesized (Table 5).

TABLE 5
The guide RNAs and ssDNA reporter used for CRISPR

| | Sequence (5′-3′) |
|---|---|
| SARS-COV- | UAAUUUCUACUAAGUGUAGAUCUUAUGCA |
| 2_nsp8_gRNA | UCAGCAUUGUGGGA (SEQ ID NO: 1) |
| SARS-COV- | UAAUUUCUACUAAGUGUAGAUCCCCCAGC |
| 2_N_gRNA | GCUUCAGCGUUC (SEQ ID NO: 2) |
| [9, 10] | |
| SSDNA | 6FAM-TTATTATT-IABKFQ (SEQ ID NO: 3) |
| reporter* | |

One-Tube RT-LAMP-CRISPR Assays for SARS-CoV-2 Detection

A 10 μL of the RT-LAMP reagent mixture was added to the bottom of a 0.5 mL PCR tube (Sarstedt, Germany) and 10 μL of the Cas12a reagent mixture was added inside the cap of the tube (FIG. 1). TNA (5 μL) was added to the bottom of the tube and mixed with the RT-LAMP reagent by pipetting up and down. The tube was gently capped and put in a thermocycler (Eppendorf, Germany) without closing the lid, and the bottom of the tube was kept at the optimal temperature for 30-40 minutes for RT-LAMP reaction. When RT-LAMP reaction was completed, the tube was flicked by wrist and mixed the Cas12a reagent mixture with the RT-LAMP amplicon. The tube was then incubated in the thermocycler at a constant temperature of 37° C. for 10 minutes. Samples that showed green fluorescence were regarded as positive under the excitation of a UV lamp, and were images were taken. The fluorescence intensity was also measured by a Qubit 4 Fluorometer (Invitrogen, USA). Samples that showed the fluorescence intensity above the cut-off values were regarded as positive.

Real-Time RT-PCR Assay for SARS-CoV-2 Detection

The LightMix® SarbecoV E-gene kit (TIB Molbiol, Germany) with LightCycler Multiplex RNA Virus Master (Roche) was used according to the manufacturer's instructions. Each 20 μL reaction mixture consisted of 5.4 μL of nuclease-free water, 0.5 μL of reagent mix, 4 μL of Roche Master, 0.1 μL of RT enzyme and 10 μL of TNA as the template. RT-PCR was performed using a LightCycler 480 II Real-Time PCR System (Roche). The thermal cycling condition was 55° C. for 3 minutes, 95° C. for 30 seconds, followed by 45 cycles of 95° C. for 3 seconds and 60° C. for 12 seconds.

Statistical Analysis

Kappa statistics was used to determine the agreement between the in-house developed assay and the reference method. McNemar's test was used to compare the performance of the in-house developed assay with the reference method. Fluorescence readings of the two RT-LAMP-CRISPR assays were compared using Wilcoxon signed-rank test. Spearman's correlation was used to assess the relation between the fluorescence readings of RT-LAMP-CRISPR assays and the Cp values of the LightMix E-gene RT-PCR. P<0.05 was considered statistically significant. Statistical analysis was performed using GraphPad PRISM® 9 or IBM SPSS Statistics 26.

Results

Design of a COVID-19 RT-LAMP-CRISPR Assay Targeting the Nsp8 Gene of the SARS-CoV-2 in a One-Tube Format

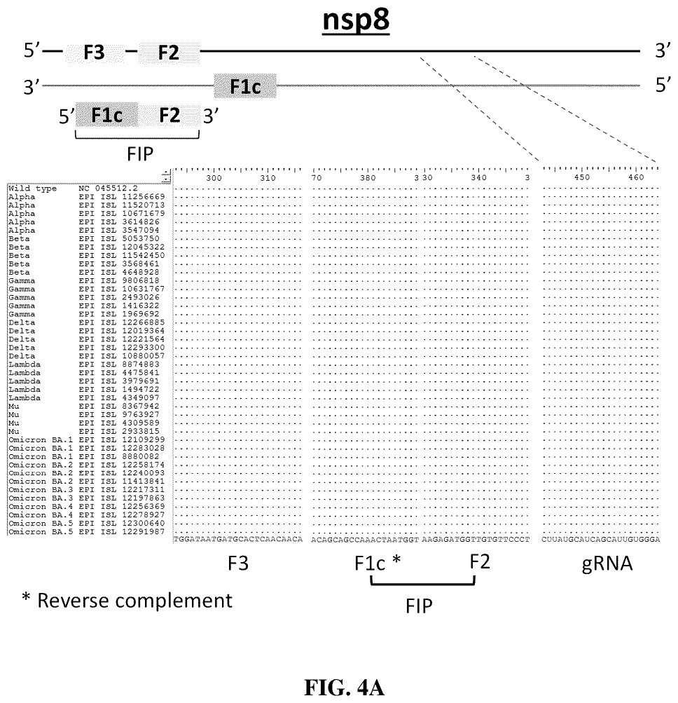

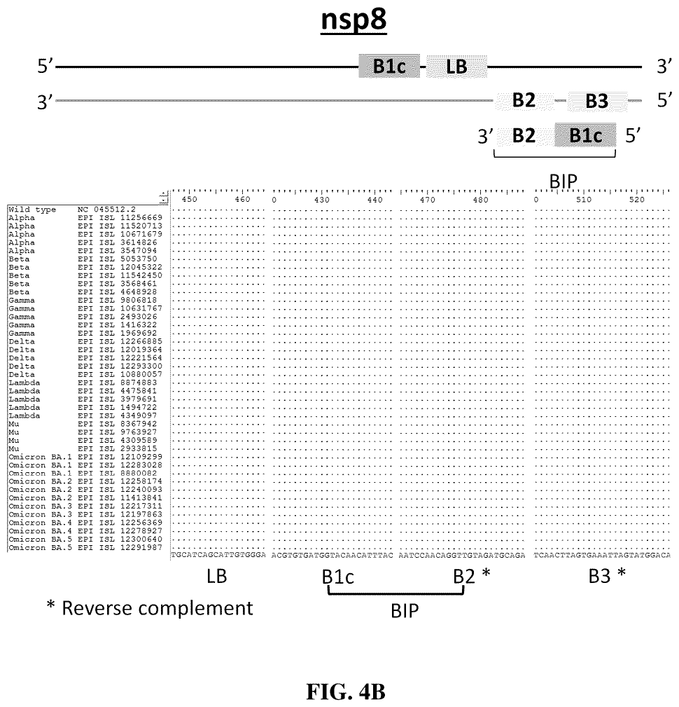

Primers were designed targeting the nsp8 gene region as a target for RT-LAMP (Tables 1 (shown above) and 6) using the New England Biolabs (NEB) LAMP Primer Design tool (available at the website: lamp.neb.com/#!/). Additional primers designed by Broughton et al.=²¹ targeting the N gene were used in a comparative RT-LAMP-CRISPR assay (Tables 1 and 6). A multiple-sequence alignment showed that the target sites of our nsp8 primers and gRNA were well conserved among different variants (ancestral strain, Alpha, Beta, Gamma, Delta, Lambda, Mu, and Omicron [BA.1, BA.2, BA.3, BA.4, and BA.5]) (FIGS. 4A and 4B).

In the present study, the one-tube-format RT-LAMP-CRISPR assay was achieved by mixing a sample nucleic acid extract with an RT-LAMP reagent mixture at the bottom of a PCR tube and adding a Cas12a reagent mixture inside the cap of the tube (24). After the RT-LAMP reaction, the Cas12a reagent mixture was flicked to the bottom of the tube and mixed with the RT-LAMP amplicon without opening the tube for CRISPR. Fluorescence could be visualized under UV excitation or measured using a fluorometer.

Analytical Sensitivity and Specificity of the Novel Nsp8 Gene and the Comparative Nucleocapsid (N) Gene RT-LAMP-CRISPR Assays for SARS-CoV-2 Detection in One-Tube Format

To compare the analytical sensitivity of the nsp8 and N gene RT-LAMP-CRISPR assays, the limit of detection (LOD) was evaluated using two-fold serial dilutions of a total nucleic acid (TNA) extracted from the SARS-CoV-2 Q control 01, a positive control with a target SARS-CoV-2 concentration of 10000 copies/mL. The LOD of the nsp8 assay was 750 copies/mL and the N assay was 2000 copies/mL (equivalent to 15 and 40 copies/reaction respectively) (Table 7). The LOD of the commercial LightMix E-gene real-time RT-PCR assay was 31.3 copies/mL (equivalent to 1.3 copies/reaction) (Table 7). Both nsp8 and N gene RT-LAMP-CRISPR assays showed no cross reaction with SARS-CoV-1 and other common respiratory viruses.

TABLE 6
N Primers^{21,22} used for RT-LAMP reactions in
this study

| | |
|---|---|
| Primer | Sequence (5′-3′) |

N gene of SARS-COV-2

| | |
|---|---|
| SARS- | AACACAAGCTTTCGGCAG (SEQ ID NO: 9) |
| CoV- | |
| 2_N_F3 | |
| SARS- | GAAATTTGGATCTTTGTCATCC (SEQ ID NO: 10) |
| CoV- | |
| 2_N_B3 | |
| SARS- | TGCGGCCAATGTTTGTAATCAGCCAAGGA |
| CoV- | AATTTTGGGGAC (SEQ ID NO: 11) |
| 2_N_FIP | |
| SARS- | CGCATTGGCATGGAAGTCACTTTGATGGC |
| CoV- | ACCTGTGTAG (SEQ ID NO: 12) |
| 2_N_BIP | |
| SARS- | TTCCTTGTCTGATTAGTTC (SEQ ID NO: 13) |
| CoV- | |
| 2_N_LF | |
| SARS- | ACCTTCGGGAACGTGGTT (SEQ ID NO: 14) |
| CoV- | |
| 2_N_LB | |

Human RNase P gene (21)

| | |
|---|---|
| NaseP- | TGATGAGCTGGAGCCA (SEQ ID NO: 15) |
| POP7_F3 | |
| RNaseP- | CACCCTCAATGCAGAGTC (SEQ ID NO: 16) |
| POP7_B3 | |
| RNaseP- | GTGTGACCCTGAAGACTCGGTTTTAGCCACTGACTCG |
| POP7_FIP | GATC (SEQ ID NO: 17) |
| RNaseP- | CCTCCGTGATATGGCTCTTCGTTTTTTTCTTACATGG |
| POP7_BIP | CTCTGGTC (SEQ ID NO: 18) |
| RNaseP- | ATGTGGATGGCTGAGTTGTT (SEQ ID NO: 19) |
| POP7_LF | |
| RNaseP- | CATGCTGAGTACTGGACCTC (SEQ ID NO: 20) |
| POP7_LB | |

TABLE 7
Test results for determining the limit of detection of the
in-house developed nsp8 and N gene RT-LAMP-CRISPR assays
and the commercial LightMix E gene RT-PCR assay with TNA
extracted from a Qnostics SARS-CoV-2 Q Control 01

| Concentration | Intra-run | | | Inter-run | | |
|---|---|---|---|---|---|---|
| (copies/mL) | Test 1 | Test 2 | Test 3 | Test 1 | Test 2 | Test 3 |

nsp8 gene RT-LAMP-CRISPR assay

| | | | | | | |
|---|---|---|---|---|---|---|
| 1000 | + | + | + | + | + | + |
| 750 | + | + | + | + | + | + |
| 500 | + | + | + | − | − | − |
| 250 | + | − | − | − | − | − |

N gene RT-LAMP-CRISPR assay

| | | | | | | |
|---|---|---|---|---|---|---|
| 4000 | + | + | + | + | + | + |
| 2000 | + | + | + | + | + | + |
| 1000 | + | + | + | + | + | − |
| 500 | − | − | − | + | + | + |

LightMix E gene RT-PCR assay

| | | | | | | |
|---|---|---|---|---|---|---|
| 125 | + | + | + | + | + | + |
| 62.5 | + | + | + | + | + | + |
| 31.3 | + | + | + | + | + | + |
| 15.6 | + | − | − | + | + | − |
| 125 | + | + | + | + | + | + |

Evaluation of the Diagnostic Performance of the Nsp8 and N Gene RT-LAMP-CRISPR Assays for SARS-CoV-2 Detection Using Sample Extracts

To assess the diagnostic performance of the nsp8 and N gene RT-LAMP-CRISPR assays in clinical specimens, a total of 319 respiratory specimens from suspected COVID-19 patients that were previously tested by the commercial LightMix E-gene RT-PCR assay were selected. Among these specimens, 46.4% (148/319) tested positive for SARS-CoV-2 by the LightMix E-gene RT-PCR (median Cp:23.1; range: 14.2-36.6), while 45.8% (146/319) tested positive for SARS-CoV-2 by both nsp8 and N gene RT-LAMP-CRISPR assays (Table 8). There was no significant difference in the detection rate between the in-house nsp8/N gene RT-LAMP-CRISPR assay and the commercial E-gene RT-PCR assay (p=0.5) (Table 8). To monitor the presence of cellular mate-rial in the clinical specimens, primers targeting the human RNase P gene (Table 1) were used as internal controls (21). Fluorescence was detected for all of the available sample extracts by the RNase P RT-LAMP-CRISPR assay, proving the validity of the tested specimens (data not shown). Using the LightMix E-gene RT-PCR assay as the reference method, the diagnostic sensitivity and specificity of both nsp8 and N gene RT-LAMP-CRISPR assays were 98.6% and 100%, respectively. The sensitivity of both the nsp8 and N gene RT-LAMP-CRISPR assays using nasopharyngeal specimens (median Cp, 21.8 [range, 14.2 to 33.5]) was 100%, while that of both assays using saliva specimens (median Cp, 24.7 [range, 14.6 to 36.6]) was 97.1% (Table 8). All 122 specimens with moderate to high viral load (Cp<30) tested positive for SARS-CoV-2, while 92.3% (24/26) specimens with low viral loads (Cp≥30) tested showed positive for SARS-CoV-2 for the specimens (Table 9).

TABLE 8
Diagnostic performance of the in-house developed nsp8
and N gene RT-LAMP-CRISPR assays compared to the LightMix
E-gene RT-PCR assay for SARS-CoV-2 detection

| Molecular | LightMix E-gene RT-PCR | | Kappa Value | McNemar's |
|---|---|---|---|---|
| assays | Positive | Negative | (95% CI)* | Test |

nsp8 RT-LAMP-CRISPR

| | | | | |
|---|---|---|---|---|
| Positive | 146 | 0 | 0.987 | P = 0.5 |
| Negative | 2 | 171 | (0.97-1) | |

N RT-LAMP-CRISPR

| | | | | |
|---|---|---|---|---|
| Positive | 146 | 0 | 0.987 | P = 0.5 |
| Negative | 2 | 171 | (0.97-1) | |

TABLE 9
Test performance of the in-house nsp8
and N gene RT-LAMP-CRISPR assays

| In-house RT- | Sensitivity | | | |
|---|---|---|---|---|
| LAMP-CRISPR | Cp ≤ 25 | Cp >25 − <30 | Cp ≥ 30 | Overall |
| Assays | (n = 89) | (n = 33) | (n = 26) | (n = 148) |
| nsp8 assay | 100% | 100% | 92.3% | 98.6% |
| N assay | 100% | 100% | 92.3% | 98.6% |

The lineages of SARS-CoV-2 in the 146 positive specimens were identified by nanopore sequencing. The isolates belonged to Variants of Concern (Alpha, Beta, Gamma, Delta, and Omicron) and variants being Monitored (Eta, Kappa, Mu and Theta) (Tables 10 and 11). The nsp8 and N gene RT-LAMP-CRISPR assays of the present study were able to detect the recently emerged Omicron variants as evidenced by green fluorescence for the eight clinical specimens (median Cp: 24.3; range 15.8-32.5) The Omicron variants were detected by the nsp8 and N gene RT-LAMP-CRISPR assays in which lanes 1-8 contained the clinical specimens containing omicron variants confirmed by nanopore sequencing, lane 9 contained the negative control and lane 10 contained the positive control (data not shown). For the two specimens that tested negative by the nsp8 and N RT-LAMP-CRISPR assays but positive by the LightMix E-gene RT-PCR, the lineage of SARS-CoV-2 could not be identified, which was probably due to low viral load that was revealed by high Cp values (36.2 and 36.6) in these two specimens. Among the three proficiency test (PT) samples from the College of American Pathologists (CAP) and 8 PT samples from Quality Control for Molecular Diagnostics (QCMD), both nsp8 and N gene RT-LAMP-CRISPR assays provided 100% accurate results.

TABLE 10
Variants of SARS-CoV-2 identified in clinical specimens

| VOC/VBM* | Number |
|---|---|
| Alpha | 10 |
| Beta | 8 |
| Gamma | 1 |
| Delta | 40 |
| Omicron | 8 |
| Eta | 3 |
| Kappa | 13 |
| Mu | 1 |
| Theta | 2 |

*Variants of Concern/Variants being Monitored (from WHO)

TABLE 11
Lineages of SARS-CoV-2 identified in clinical specimens

| Pango | Number |
|---|---|
| A | 1 |
| A.21 | 1 |
| AY.3 | 1 |
| B | 1 |
| B.1 | 5 |
| B.1.1 | 3 |
| B.1.1.529 | 8 |
| B.1.1.63 | 6 |
| B.1.1.7 | 10 |
| B.1.177 | 2 |
| B.1.210 | 1 |
| B.1.351 | 7 |
| B.1.351.3 | 1 |
| B.1.36 | 5 |
| B.1.36.27 | 19 |
| B.1.459 | 3 |
| B.1.466.2 | 5 |
| B.1.470 | 3 |
| B.1.480 | 1 |
| B.1.525 | 3 |
| B.1.562 | 1 |
| B.1.617.1 | 13 |
| B.1.617.2 | 39 |
| B.1.621 | 1 |
| B.43 | 1 |
| B.6 | 2 |
| P.1 | 1 |
| P.3 | 2 |
| Untypeable | 2 |

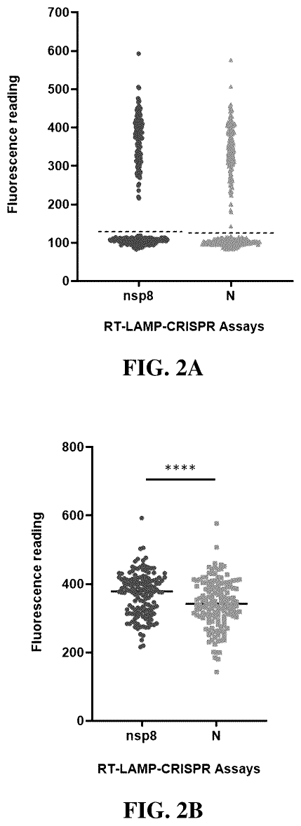

For qualitative detection of SARS-CoV-2 RNA in this study, the green fluorescence was visualized under UV excitation and measured fluorescence intensity by a fluorometer for each reaction. The cut-off of fluorescence measured by the fluorometer was determined by calculating the mean+3 standard deviation of the fluorescence readings of the specimens that tested negative by LightMix E-gene RT-PCR. The cut-off values for the nsp8 and N gene RT-LAMP-CRISPR assays were 126.3 (FIG. 2A) and 123.2 (FIG. 2B), respectively. Among the 319 clinical specimens, the fluorescence readings of the 146 specimens that tested positive by the LightMix E-gene RT-PCR were above the cut-off values and those of the 171 specimens that tested negative by the E-gene RT-PCR were below the cut-off values. For the two specimens that tested positive by the E-gene RT-PCR but negative by the disclosed RT-LAMP-CRISPR assays, their fluorescence readings were between 96.9 and 107. Among the 146 clinical specimens that tested positive by our RT-LAMP-CRISPR assays, the median fluorescence reading generated from the nsp8 assay (378.3; range: 215.6-592.6) was significantly higher than that from the N assay (342.0; range: 143.0-576.6) (P<0.0001) (FIGS. 2A and 2B). Green fluorescence could also be easily visualized with the naked eye for both naso-pharyngeal and saliva specimens under UV excitation (data not shown).

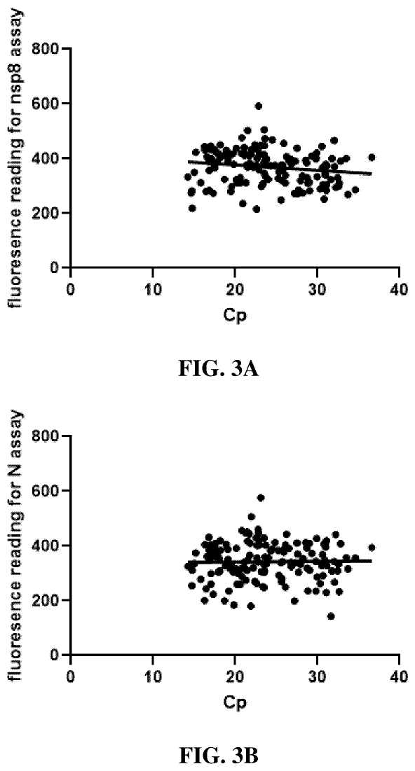

To determine if there is any correlation between the fluorescence readings of RT-LAMP-CRISPR assays and the Cp values of the LightMix E-gene RT-PCR, scatterplots were drawn (FIG. 3A and FIG. 3B). A weak correlation was noted between the fluorescence readings of the nsp8 assay and the Cp values of E-gene RT-PCR (r=−0.2; P=0.02), while there was no correlation between the fluorescence readings of the N gene RT-LAMP-CRISPR assay and the Cp values of LightMix E-gene RT-PCR (r=0.04; P=0.61).

Diagnostic Performance of RT-LAMP-CRISPR Assays without Nucleic Acid Extraction

As viral nucleic acid extraction is an extra step that takes time and requires specialized skill, the disclosed RT-LAMP-CRISPR assays were tested using nasopharyngeal swab (NPS) and saliva specimens directly without nucleic acid extraction. First, the optimal sample treatment and sample volume required for the reaction was determined. The data showed that the RT-LAMP-CRISPR assay was negative when the volume of the NPS specimen was 5 mL, while 1 mL of the NPS specimen without heat pretreatment and 1 mL of saliva with heat pretreatment (98° C. for 5 min) were the optimal conditions for the disclosed RT-LAMP-CRISPR assays (data nor shown).

Next, a total of 28 specimens were evaluated, including 14 NPS and 14 saliva specimens. The sensitivities of the nsp8 or N gene RT-LAMP-CRISPR assay were 27.3% (3/11) for NPSs and 54.5% (6/11) for saliva. All specimens that tested negative by RT-PCR also tested negative by the nsp8 or N gene RT-LAMP-CRISPR assay. The specimens that tested positive by the nsp8 or N gene RT-LAMP-CRISPR assay had real-time RT-PCR Cp values of #22.3 for NPS and #25.8 for saliva specimens.

### Discussion

The present study describes the low-cost one-tube RT-LAMP-CRISPR assays for COVID-19 diagnosis. The disclosed one-tube format is made possible by depositing reagents in the lid of the vial, and therefore, does not require opening of the tube during the assay process, which significantly reduces the risk of contamination due to amplicon carryover. Several studies have described similar approaches with similar sensitivities and specificities, but the reagent costs of our RTLAMP-CRISPR assays, including enzymes, primers, deoxynucleoside triphosphate (dNTP), MgSO₄, Cas12a protein, gRNA, and the single-stranded DNA (ssDNA) reporter, were less than $2 per reaction, which is lower than the costs of the RT-LAMP-CRISPR assays reported by others since smaller amounts of RT-LAMP reagents were used in our study. RT-LAMP reagents are quite inexpensive, unlike those for other isothermal amplification formats like recombinase polymerase amplification (RPA) assays for which a commercial kit is not available and that require the addition of reverse transcriptase from another source, increasing the costs. The assay described herein is superior to previously described assays at least because it lowers the cost associated with the assay by reducing RT-LAMP reagent volumes used per reaction, with RT-LAMP and CRISPR reagents costing less than U.S. $2 per reaction^{22, 23, 24}. Reagent costs are quite cheap with the assay disclosed herein, unlike other isothermal amplification formats like recombinase polymerase amplification (RPA) assays for which a commercial kit of RT-RPA is not available and requires addition of a reverse transcriptase from another source, increasing costs^{25, 26}.

Primers targeting the nsp8 and N genes for used for two separate RT-LAMP-CRISPR assays. Due to the ongoing emergence of new variants with mutations scattered across the genome, it is always advisable to design primers targeting different genes. The SARS-CoV-2 N gene is a common target for COVID-19 NAAT assays, but N gene target failure has been reported^{27, 28}. FIG. 4A and FIG. 4B are schematic representations of the multiple sequence alignment using the presently described primer and gRNA sequences and the nsp8 gene sequences of SARS-CoV-2 variants (wild type, Alpha, Beta, Gamma, Delta, Omicron, Lambda and Mu) from different geographical regions. Wild type sequence (NC_045512.2) and the sequences of other variants were obtained from GenBank and GISAID, respectively. The disclosed assays could detect all evaluated variants of concern (including the Omicron variant) and variants under investigation. In the present study showed that the median fluorescence reading from the nsp8 assay was significantly higher than that of the N-gene assay, demonstrating that the nsp8 assay allows better visualization, especially for the samples with low viral load when observing by naked eye.

The disclosed RT-LAMP-CRISPR assays were evaluated using sample extracts, but nucleic acid extraction takes time and requires specialized skill to perform. Therefore, the assay was performed on direct clinical specimens to test our assays. During assay optimization, no fluorescence was detected for the reaction using 5 mL of a direct specimen (the same volume as that for the viral nucleic acid that we used for testing); this may be due to the presence of inhibitors in clinical specimens when larger sample volumes were used. A stronger signal could be detected when smaller volumes (1 or 2 mL) of direct specimens were used. The assays did not use lysis buffer/proteinase K in this evaluation because of the extra cost and inconvenience compared to heat pretreatment, so we only compared the samples with and without heat pretreatment. Heat pretreatment improved SARS-CoV-2 detection for saliva specimens but not for NPS specimens. This finding was consistent with those of another study that demonstrated a significant increase in the detection sensitivity when using saliva samples with prolonged heat pretreatment (33). Finally, 1 mL of NPSs without heat pretreatment and 1 mL of saliva with heat pretreatment at 98° C. for 5 min were the optimal conditions for RT-LAMP-CRISPR assays. Studies further evaluated 14 NPS and 14 saliva specimens with various concentrations of SARS-CoV-2 or that were negative for SARSCoV-2 by the disclosed RT-LAMP-CRISPR assays using the optimized conditions. Although a lower detection sensitivity was noted for the assays using direct specimens than for the assays using purified sample extracts, it is interesting to note that our assays using direct saliva specimens with heat pretreatment showed a higher detection sensitivity than that of assays using direct NPS specimens. Nevertheless, the use of direct specimens can help reduce the time and cost compared to viral nucleic acid extraction, and it is particularly useful when there is a shortage of extraction reagents.

Although real-time RT-PCR is the most common method for COVID-19 diagnosis due to its high sensitivity and specificity, RT-LAMP-CRISPR assays carry considerable advantages over RT-PCR assays. The disclosed RT-LAMP-CRISPR assays performed with comparable sensitivity and specificity to the real-time RT-PCR assay. These assays do not require real-time PCR systems which are bulky and expensive. Indeed, they can be performed on simple heating blocks^{21, 22}. The equipment cost of real-time RT-PCR is >45-fold higher than that of RT-LAMP. Furthermore, the running time of RT-LAMP-CRISPR assays is less than an hour, which is shorter than RT-PCR assays²⁹⁻³². The fluorescent readout enables visual interpretation of results, although this can be enhanced by a fluorometer. The advantage of using a fluorometer over the naked eye is that measurement by a fluorometer is an objective readout of the fluorescence, which eliminates the subjectiveness of using the naked eye.

The RT-LAMP-CRISPR assay (fluorometric approach) has several advantages over the colorimetric RT-LAMP assay. First, the colorimetric assay has a higher chance of giving false-positive results than the fluorometric assay. The colorimetric assay relies on the change in the pH, and therefore, a specimen with a lower pH can lead to false-positive results in the colorimetric assay. Second, elution buffers for viral RNA extraction could significantly affect colorimetric readings, such as false-negative results; this may be due to the pH effect of or the chemicals in these buffers, while the elution buffer that we used for extraction did not have an adverse effect on our RT-LAMP CRISPR assays. Third, colorimetric interpretation was time sensitive for samples, including the negative control, which could turn positive when the incubation time was more than 40 min for the RT-LAMP reaction. Fourth, the colorimetric assay does not involve the use of gRNA, and thus, there is a higher chance of the detection of nonspecific products. To overcome this problem, the addition of Cas protein and gRNA specific for a viral gene target can enhance the specificity of the fluorometric assay. Fifth, a fluorometric assay with higher detection sensitivity than a colorimetric assay has been reported.

False positive results by RT-LAMP can be due to spurious amplification or pH effect of clinical specimens³³⁻³⁶. To overcome this problem, addition of Cas protein and gRNA specific to a viral gene target can enhance specificity. Cas12b has previously been used for CRISPR reaction in several studies, but it requires >100 nucleotide(nt)-long gRNA for the reaction^{37, 38}. Since longer gRNA has a risk of partial overlap between the gRNA and one of the primers for RT-LAMP, this may lead to false positive results due to sporadic collateral activity^{22, 37}. Besides, the cost of longer synthetic RNA is more expensive. In the present study, Cas12a was used for CRISPR which requires around 40 nt-long gRNA for the reaction. Hence, the chance of false positive results can be reduced. Cas12a has distinct advantages over other Cas-associated proteins used for diagnostic applications, such as Cas3, Cas9, Cas12b, or Cas13a (44-48). Cas12b was used for CRISPR reactions in several studies, but it requires >100-nucleotide (nt)-long gRNA for the reaction. Since longer gRNA has a risk of partial overlap between the gRNA and one of the primers for RTLAMP, this may lead to false-positive results due to sporadic collateral activity. Besides, the cost of longer synthetic RNA is higher. Cas12a for CRISPR requires; 40-nt-long gRNA for the reaction. Hence, the chance of false positive results can be reduced. Moreover, the Cas12a protein is commercially available. Furthermore, unlike Cas13a, Cas12a does not require the additional transcription of the DNA into RNA.

In summary, the presently described RT-LAMP-CRISPR assays for detecting SARS-CoV-2 nsp8 and N genes are sensitive, specific, affordable, fast, and easy to perform. As the COVID-19 pandemic continues, it is believed that such assays have considerable value in resource limited settings to improve COVID-19 diagnostic capacity.

The present invention can be further understood by reference to the following paragraphs.
1. A nucleic acid composition detecting the presence of SARS-CoV-2 nsp8 gene in a sample comprising or consisting of a nucleic acid sequence SEQ ID NO:4; SEQ ID NO:5, SEQ ID NO:5, SEQ ID NO:7, and SEQ ID NO:8, or a nucleic acid sequence having at least 70%, 75%, 80%, 85%, 90%, 95%, 96%, 97%, 98%, or 99% sequence identity to any of the foregoing.
2. The composition of paragraph 1, further comprising (a) a Cas/sgRNA pair, and (b) one or more fluorescent reporters, one or more quenchers, or a combination thereof.
3. The composition of any one of paragraphs 1-2, comprising suitable buffer (such as a phosphate buffer or Tris buffer).
4. The composition of any one of paragraphs 1-3, comprising one or more salts.
5. The composition of paragraph 4, wherein the salt is a potassium, sodium, magnesium or ammonium salt.
6. The composition of paragraph 5, wherein the salt is selected from the group consisting of KCl, NaCl, MgCl₂, MgSO₄, (NH₄)₂SO₄).
7. The composition of any one of claims 1-6, further comprising one or more detergents.
8. The composition of any one of paragraphs 1-7, comprising one or more nucleotides or nucleotide analogues.
9. The composition of any one of paragraphs 1-8, further comprising one or more of dATP, dCTP, dGTP, and dTTP.
10. The composition of any one of paragraphs 1-9, further comprising a reverse transcriptase and a DNA polymerase with strand displacement activity.
11. The composition of any one of paragraphs 2-9, wherein the sgRNA comprises SEQ ID NO:1 or a nucleic acid sequence having at least 70%, 75%, 80%, 85%, 90%, 95%, 96%, 97%, 98%, or 99% sequence identity to any of the foregoing.
12. The method of paragraph 11, wherein the reporter nucleic acid comprises SEQ ID NO:3, or a nucleic acid sequence having at least 70%, 75%, 80%, 85%, 90%, 95%, 96%, 97%, 98%, or 99% sequence identity to any of the foregoing.
13. A method of detecting presence of SARS-CoV-2 nucleic acid in a sample, comprising the steps of:
   (a) contacting the sample with a set of reverse transcription-loop-mediated isothermal amplification (RT-LAMP) primers specific for the nsp8 gene of SARS-CoV-2 under conditions sufficient for amplification of the nsp8 gene of SARS-CoV-2, wherein the set of RT-LAMP primers and (b) detecting the SARS-CoV-2 nsp8 gene amplification product, thereby detecting presence of SARS-CoV-2 nsp8 in the sample, the method including an optional step before step (b) of contacting the sample from step (a) with a composition comprising a Cas enzyme/sgRNA pair and a reporter nucleic acid.
14. The method of paragraph 13, comprising contacting the sample with the composition of any one of paragraphs 1-10.
15. The method of paragraph 13, wherein the primers comprise SEQ ID Nos:4-8.
16. The method of any one of paragraphs 13-15, wherein the primers comprise a nucleic acid sequence having at least 90% sequence identity to any one of SEQ ID NOs: 4-8.
17. The method of any one of paragraphs 13-14, wherein the sample is selected from the group consisting of mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood, wherein the method comprises a step of extracting nucleic acids from the sample and contacting the sample with the RT-LAMP primers.
18. The method of paragraph 17, wherein the sample is obtained from a nasopharyngeal swab, a nasopharyngeal aspirate, sputa/deep throat saliva or a throat swab.
19. The method of any one of paragraphs 13-18, wherein the RT-LAMP reaction conditions comprise a reaction temperature of about 60° C. and primer final concentrations: SEQ ID NO:4; SEQ ID NO:5, from about 0.1 to about 0.4 μM, preferably, about 0.2 μM), (SEQ ID NO:6: from about 0.8 to about 3.2 μM; SEQ ID NO:7: from about 0.8 to about 3.2 μM, preferably, about 1.6 μM), and SEQ ID NO:8; from about 0.2 to about 1.6 μM, preferably, about 0.4 μM.
20. The method of any one of paragraphs 13-19, wherein the RT-LAMP reaction comprises a reverse transcriptase, DNA polymerase, pH indictor, and six primers to amplify the SARS-CoV-2 E gene, causing a drop in pH and a color change from pink to yellow, indicating the presence of the SARS-CoV-2 in the sample.
21. A Kit the presence of SARS-CoV-2 nsp8 gene in a sample comprising the composition of any one of paragraphs 1-12 in one or more containers.
22. The kit of paragraph 21, wherein one or more components of the composition are lyophilized.
23. The kit of paragraph 21 or 22, comprising two containers, wherein one container comprises lyophilized components, and the second container comprise a rehydration solution comprising primers for RT-LAMP and a ssDNA reporter.
24. The kit of paragraph 22 or 23 comprising RT-LAMP reaction reagents except primers at the bottom on a container and Cas enzyme reaction regents on the lid of the container, wherein (a) the RT-LAMP reaction reagents are selected from the group consisting of dNTP, isothermal amplification buffer, MgSO4, RNase inhibitor, reverse transcriptase, and DNA polymerase, and a suitable stabilizing agent such as D-(+)-trehalose dihydrate, mannitol, sucrose; and (b) Cas enzyme reaction reagents comprise an RNP complex for the Cas12a-mediated reaction, MgSO₄, and Tris-HCl.

### REFERENCES

1. Chan, et al. 2020. Lancet 395(10223): pages 514-523, doi: 10.1016/S0140-6736(20)30154-9.
2. Guan, et al. China Medical Treatment Expert Group for Covid-19. 2020. New England Journal of Medicine Vol. 382: pages 1708-1720, DOI: 10.1056/NEJMoa2002032.
3. Zhu, et al. 2019. N Engl J Med Vol 382: pages 727-733, DOI: 10.1056/NEJMoa2001017.
4. WHO. 2022. Website: who.int/emergencies/diseases/novel-coronavirus-2019.
5. Petersen, et al. 2020. Lancet Infectious Diseases Vol. 20(9): pages e238-e244, DOI: 10.1016/S1473-3099(20)30484-9
6. Cheng, et al. 2021. Lancet Reg Health West Pac Vol. 17: article 100281, DOI:10.1016/j.lanwpc.2021.100281
7. Cheng, et al. 2020 Infect Control Hosp Epidemiol Vol. 41(11): pages 1258-1265, doi: 10.1017/ice.2020.282
8. Hung, et al. 2020. Lancet Infect Dis 20:1051-1060.
9. Hung, et al. 2020. Lancet Vol. 395(10238): pages 1695-1704, DOI:10.1016/S0140-6736(20)31042-4
10. U.S. Food & Drug Administration. 2022. fda.gov/medical-devices/coronavirus-disease-2019-covid-19-emergency-use-authorizations-medical-devices/in-vitro-diagnostics-euas.
11. Albert, et al. 2021. Clin Microbiol Infect Vol. 27(3):472.e7-472.e10.
12. Young, et al. 2020. J Clin Microbiol Vol. 59(1):e02338-20, doi: 10.1128/JCM.02338-20.
13. Babady, et al. 2021. J Mol Diagn Vol. 23: pages 3-9, doi: 10.1016/j.jmoldx.2020.10.018.
14. Poljak, et al. 2020 J Clin Microbiol Vol. 58: article e00599-20, doi: 10.1128/JCM.00599-20.
15. van Kasteren P B, et al. 2020. J Clin Virol Vol. 128: article 104412, doi: 10.1016/j.jcv.2020.104412
16. Notomi T, et al. 2015. J Microbiol Vol. 53: pages 1-5 (2015).
17. Li L, et al. 2019. ACS Synth Biol Vol. 8: pages 2228-2237, doi: 10.1021/acssynbio.9b00209.
18. Hille F, et al. 2018. Cell 172: pages 1239-1259, doi: 10.1016/j.cell.2017.11.032.
19. Li S Y, et al. 2018. Cell Res Vol. 28(4): pages 491-493, doi: 10.1038/s41422-018-0022-x
20. Chan W M et al. 2020. J Med Virol Vol. 92(1): pages 2725-2734, doi: 10.1002/jmv.26140.
21. Broughton J P, et al. 2020. Nat Biotechnol Vol. 38: pages 870-874, doi: 10.1002/jmv.26140
22. Pang B, et al 2020. Anal Chem Vol. 92: pages 16204-16212, doi: 10.1021/acs.analchem.0c04047.
23. Chen Y, et al. 2020. Biosens Bioelectron Vol. 169: article 112642, doi: 10.1016/j.bios.2020.112642.
24. Ooi K H, et al. 2021. Nat Commun Vol. 12: article 1739, doi: 10.1038/s41467-021-21996-6.
25. Feng W, et al. 2021. Anal Chem Vol. 93: pages 12808-12816, doi: 10.1038/s41467-021-21996-6.
26. de Puig H, et al. 2021. Sci Adv Vol. 7: article eabh2944, doi: 10.1126/sciadv.abh2944.
27. Miller S, et al. 2021. Microbiol Spectr Vol. 9: article e0149421, doi: 10.1128/Spectrum.01494-21.
28. Wollschläger P, et al. 2021. Clin Microbiol Infect Vol. 27: article 1353.e1-1353.e5, doi: 10.1016/j.cmi.2021.05.025.
29. Chan J F, et al. 2020 J Clin Microbiol Vol. 58: pages e00310-20, doi: 10.1128/JCM.00310-20.
30. Yip C C, et al. 2020. Int J Mol Sci Vol. 21: article 2574, doi: 10.3390/ijms21072574.
31. Brown J R, et al. 2021. J Virol Methods Vol. 295: article 114215, doi: 10.1016/j.jviromet.2021.114215.
32. van Kasteren P B, et al. 2020. J Clin Virol Vol. 128: article 104412, doi: 10.1016/j.jcv.2020.104412.
33. Dao Thi V L, et al. 2020. Sci Transl Med Vol. 12: article eabc7075, doi: 10.1126/scitranslmed.abc7075.
34. Lalli M A, et al. 2021. Clin Chem Vol. 67(2): pages 415-424, doi: 10.1093/clinchem/hvaa267.
35. Meagher R J, et al. 2018. Analyst Vol. 143(8): pages 1924-1933, doi: 10.1039/c7an01897e.
36. Uribe-Alvarez C, et al. 2021. PLoS One Vol. 16(5): article e0250202, doi: 10.1371/journal.pone.0250202.
37. Joung J, et al. 2020. N Engl J Med Vol. 383: pages 1492-1494 doi: 10.1056/NEJMc2026172.
38. Nguyen L T, et al. 2022. EBioMedicine Vol. 77: article 103926, DOI:10.1016/j.ebiom.2022.103926
39. Yoshimi K, et al. 2022. iScience Vol. 25: article 103830, doi: 10.1016/j.isci.2022.103830.
40. Marsic T, et al. 2021. Nano Lett Vol. 21(8): pages 3596-3603, doi: 10.1021/acs.nanolett.1c00612.
41. Fozouni P, et al. 2021. Cell Vol. 184(2): pages 323-333.e9, doi: 10.1016/j.cell.2020.12.001.
42. Yip C C, et al. 2020. J Clin Virol Vol. 129: 104476, doi: 10.3390/ijms21165674
43. Yip C C, et al. 2020. Int J Mol Sci Vol. 21(16): article 5674.
44. Yip C C Y, et al. 2021. Expert Rev Mol Diagn Vol. 21: pages 741-747, doi: 10.1080/14737159.2021.1933445.
45. Chan W M, et al. 2021. Lancet Reg Health West Pac Vol. 10: article 100130, DOI:10.1016/j.lanwpc.2021.100130.
Yip, et. al. J Clin Microbiol 58:e02127-19 (2020).
Ooi et al. Nat Commun 12:1739 (2021).
Rezaei, et al. Biosensors (Basel) 11:369 (2021).
Chu et al. J Clin Virol 129:104519 (2020).
Bruce e t al., PLoS Biol 18:e3000896 (2020)
Fomsgaard et al, Euro Surveill 25:2000398 (2020)
Morals et al. Adv Virol 2022:7442907 (2022).
Chow et al. Int J Mol Sci 21:5380 (2020).
Huang et al. Microb Biotechnol 13:950-961 (2020)
Wang et al. Biosens Bioelectron 172:112766 (2021).

We claim:

1. A nucleic acid composition for detecting the presence of SARS-CoV-2 nsp8 gene in a sample comprising or consisting of a nucleic acid sequence SEQ ID NO:4; SEQ ID NO:5, SEQ ID NO:5, SEQ ID NO:7, and SEQ ID NO:8, or
a nucleic acid sequence having at least 70%, 75%, 80%, 85%, 90%, 95%, 96%, 97%, 98%, or 99% sequence identity to any of the foregoing.

2. The composition of claim 1, further comprising (a) a Cas/sgRNA pair, and (b) one or more fluorescent reporters, one or more quenchers, or a combination thereof, (a) a phosphate buffer or Tris buffer, (d) a reverse transcriptase and a DNA polymerase with strand displacement activity, and/or (e) one or more detergents.

3. The composition of claim 1 comprising one or more salts.

4. The composition of claim 3, wherein the salt is a potassium, sodium, magnesium or ammonium salt.

5. The composition of claim 4, wherein the salt is selected from the group consisting of KCl, NaCl, MgCl₂, MgSO₄, (NH₄)₂SO₄).

6. The composition of claim 1, comprising one or more nucleotides or nucleotide analogues selected from the group consisting of dATP, dCTP, dGTP, and dTTP.

7. The composition of claim 2, wherein the sgRNA comprises SEQ ID NO:1 or a nucleic acid sequence having at least 70%, 75%, 80%, 85%, 90%, 95%, 96%, 97%, 98%, or 99% sequence identity to any of the foregoing.

8. The composition of claim 7, wherein the reporter nucleic acid comprises SEQ ID NO:3, or a nucleic acid sequence having at least 70%, 75%, 80%, 85%, 90%, 95%, 96%, 97%, 98%, or 99% sequence identity to any of the foregoing.

9. A method of detecting presence of SARS-CoV-2 nucleic acid in a sample, comprising the steps of:
(a) contacting the sample with a composition comprising the composition of claim 1 under conditions sufficient for amplification of the nsp8 gene of SARS-CoV-2, wherein the set of RT-LAMP primers and
(b) detecting the SARS-CoV-2 nsp8 gene amplification product, thereby detecting presence of SARS-CoV-2 nsp8 in the sample, the method including an optional step before step (b) of contacting the sample from step (a) with a composition comprising a Cas enzyme/sgRNA pair and a reporter nucleic acid.

10. The method of claim 9, wherein the primers comprise SEQ ID Nos:4-8.

11. The method of claim 9, wherein the primers comprise a nucleic acid sequence having at least 90% sequence identity to any one of SEQ ID NOs: 4-8.

12. The method of claim 11, wherein the sample is selected from the group consisting of mucus, sputum (processed or unprocessed), bronchial alveolar lavage (BAL), bronchial wash (BW), bodily fluids, cerebrospinal fluid (CSF), urine, tissue (e.g., biopsy material), rectal swab, nasopharyngeal aspirate, nasopharyngeal swab, throat swab, feces, plasma, serum, or whole blood, wherein the method comprises a step of extracting nucleic acids from the sample and contacting the sample with the RT-LAMP primers.

13. The method of claim 12, wherein the sample is obtained from a nasopharyngeal swab, a nasopharyngeal aspirate, sputa/deep throat saliva or a throat swab.

14. The method of claim 13, wherein the RT-LAMP reaction conditions comprise a reaction temperature of about 60° C. and primer final concentrations: (i) SEQ ID NO:4/SEQ ID NO:5, from about 0.1 to about 0.4 μM;
(ii) SEQ ID NO:6, from about 0.8 to about 3.2 μM;
(iii) SEQ ID NO:7, from about 0.8 to about 3.2 μM; and
(iv) SEQ ID NO:8; from about 0.2 to about 1.6 μM.

15. The method of claim 14, wherein, (i) the concentration of SEQ ID NO:4/SEQ ID NO:5 is about 0.2 μM; (ii) the concentration of SEQ ID NO:7 is about 1.6 μM; and/or (iii) the concentration of SEQ ID NO:8 is about 0.4 μM.

16. The method of claim 14, wherein the RT-LAMP reaction comprises a reverse transcriptase, DNA polymerase, pH indictor, and six primers to amplify the SARS-CoV-2 E gene, causing a drop in pH and a color change from pink to yellow, indicating the presence of the SARS-CoV-2 in the sample.

17. A Kit the presence of SARS-CoV-2 nsp8 gene in a sample comprising the composition of claim 1 in one or more containers.

18. The kit of claim 17, wherein one or more components of the composition are lyophilized.

19. The kit of claim 18, comprising two containers, wherein one container comprises lyophilized components, and the second container comprise a rehydration solution comprising primers for RT-LAMP and a ssDNA reporter.

20. The kit of claim 18 comprising RT-LAMP reaction reagents except primers at the bottom on a container and Cas enzyme reaction regents on the lid of the container, wherein (a) the RT-LAMP reaction reagents are selected from the group consisting of dNTP, isothermal amplification buffer, MgSO₄, RNase inhibitor, reverse transcriptase, and DNA polymerase, and a suitable stabilizing agent such as D-(+)-trehalose dihydrate, mannitol, sucrose; and (b) Cas enzyme reaction reagents comprise an RNP complex for the Cas12a-mediated reaction, MgSO₄, and Tris-HCl.
